(19) United States
(12) Patent Application Publication
(10) Pub. No.: US 2025/0281767 A1
(43) Pub. Date: Sep. 11, 2025

(54) MULTIPURPOSE DEVICE WITH CONTROLLABLE PHOTONIC AND ELECTROMAGNETIC MODALITIES

(71) Applicant: Visibelle Derma Institute, Inc., Bloomington, MN (US)

(72) Inventor: Steven J. Daffer, Edina, MN (US)

(21) Appl. No.: 19/069,978

(22) Filed: Mar. 4, 2025

Related U.S. Application Data
(60) Provisional application No. 63/561,470, filed on Mar. 5, 2024.

(51) Int. Cl. A61N 5/06

(52) U.S. Cl. CPC A61N 5/0616; A61N 2005/0626; A61N 2005/0628; A61N 2005/0633; A61N 2005/0643; A61N 2005/0652; A61N 2005/0659; A61N 2005/0661

(57) ABSTRACT

A device for providing controlled photonic modalities includes a substrate having a length and a width, at least one array of controllable photonic emitters and at least one electromagnetic emitter both attached the substrate. A controller is configured to allow a user to select the type of photonic energy and electromagnetic energy.

### CROSS REFERENCE TO RELATED APPLICATION(S)

The present application claims the benefit of U.S. Provisional Patent Application Ser. No. 63/561,470 entitled MULTIPURPOSE DEVICE WITH CONTROLLABLE PHOTONIC AND ELECTROMAGNETIC MODALITIES that was filed on Mar. 5, 2024, the contents of which are incorporated by reference in its entirety.

### BACKGROUND

The present disclosure relates a multipurpose device that is configured for use as a mat, a cover for draping on a surface or as a hanging sheet. More particularly, the present disclosure relates to a multipurpose device having controllable photonic and/or electromagnetic modalities that can provide one or more treatments to a person, provide ambiance to a space and/or change environmental conditions within a space.

Many people desire to utilize photonic energy for treatments or to enhance the effects of exercise, physical therapy, massage and the like. However, to obtain the photonic treatments, the user typically has to travel to a location, which can be inconvenient.

Additionally, even if a user were able to obtain a device for the photonic treatment, the devices can be bulky and difficult to store. Especially where a user's living space is limited, the size of the photonic device may prevent a user from obtaining the photonic device.

The different photonic energies provide different benefits to the user. By way of non-limiting example, red light (620-750 nm) in the visible light spectrum is known to improve skin appearance, such as reducing wrinkles, scars, redness and acne and aid in recovery after working out or experiencing trauma to the person's body. Green light (525-550 nm) is known to improve the appearance of the skin by lessening dark circles, pigmentation, broken capillaries and sunspots and calm irritated or over-stimulated skin. Blue light therapy (405-410 nm) can be used to treat acne and can improve skin tone and texture. Yellow light therapy (580-590 nm) can reduce the appearance of solar lentigos. Full spectrum white light (400-700 nm) can uplift mood and enhance meditative calm.

The photonic energies also include those in the infrared spectrum (800-1200 nm) which can be used for detoxification, pain relief, reduction of muscle tension, relaxation, improved circulation, weight loss, skin purification, boosting of the immune system, aid in lowering of blood pressure and aid in recovery from physical exercise. The photonic energies also include those in the ultraviolet spectrum (280-380 nm) which can be used to treat skin disorders such as psoriasis, vitiligo, destroy bacteria and promote wound healing.

The use of electromagnetic modalities creates magnetic fields that can improve the human body's natural recovery and energy, and provide treatments or biomagnetic therapies for users. The electromagnetic modalities can be used in physical recovery, healing, to lessen pain, management of diabetes, recovery from injury, assist musculoskeletal conditions, tissue repair or inflammation, or conditions including bone fractures and degenerative conditions. Additionally, electromagnetic treatments can aid in adjusting the pH in a user's body to reestablish a balanced and substantially neutral pH.

There is a need to deliver photonic and/or electromagnetic modalities in a controlled manner with a device that is configured to cover a large and/or selected area for a desired treatment while being able to be rolled and/or folded for storage in a compact configuration.

### SUMMARY

One aspect of the present disclosure includes a device for providing controlled photonic modalities and pulsed electromagnetic frequency modalities. The device includes a substrate having a length and a width, at least one array of controllable photonic emitters attached the substrate and an electromagnetic emitter. A controller is configured to allow a user to select the type of photonic energy and electromagnetic energy.

Another aspect of the present disclosure relates to a device for providing controlled photonic modalities and pulsed electromagnetic frequency modalities. The device includes a base layer having an upper surface and a lower surface configured to engage a surface and a first internal layer supporting pulsed electromagnetic field motor, the first internal layer having a lower surface facing the upper surface of the base layer and an upper surface. The device includes a second internal layer supporting one or more infrared emitters, wherein the second internal layer having a lower surface facing the first internal layer and an upper surface and a first internal insulating layer between the first internal layer and the second internal layer. The device includes a third internal layer supporting a plurality of visible light emitters, the third internal layer having a lower surface facing the upper surface of the second internal layer and a second internal insulating layer between the second internal layer and the third internal layer. The device includes a fourth internal layer supporting a plurality of stone that emit photons when heated, the fourth internal layer having a lower surface facing the upper surface of the third internal layer and an upper surface and an upper layer having a substantially optically transparent area, the upper layer having a lower surface facing the upper surface of the fourth internal layer and an upper surface configured to engaged by a user.

### BRIEF DESCRIPTION OF THE DRAWINGS

FIG. 1 is a schematic view of a multipurpose device with controlled photonic and/or electromagnetic modalities in a stored configuration and a configuration for use.

FIG. 2 illustrates the device on a lounge chair.

FIG. 3 illustrated the device on a couch.

FIG. 4 illustrates the device on a bed.

FIG. 5 illustrates the device with a plastic covering.

FIG. 6 illustrates the device positioned over a window as a drape.

FIG. 7 illustrates the device positioned over a window as a shade.

FIG. 8 illustrates the device hanging from a wall.

FIG. 9 illustrates the device hanging from a door.

FIG. 10 illustrates a controller for the device.

FIG. 11 illustrates an embodiment where the device is hanging and used as a shower curtain.

FIG. 12 illustrates an embodiment where the device is a room divider that used to modify environmental conditions in the room.

FIG. 13 is an exploded view of the multipurpose device of FIG. 1.

### DETAILED DESCRIPTION

The present disclosure relates to a multipurpose device with controllable photonic modalities and/or electromagnetic modalities that can be utilized to provide controlled photonic energy or biometric energy to a user or to provide photonic and/or electromagnetic energy to affect the environmental conditions in a space. The device includes a substrate that can be rolled into a coil for storage or can be unrolled to provide an area where photonic and/or electromagnetic energy can be controllably emitted.

In some embodiments the device is configured to be used as a mat on a floor. In other embodiments, the device can be hung vertically from a wall, a door or a rod, such as a window curtain rod, a shower rod or a room divider rod.

By way of non-limiting example, the device can emit photonic energy in the ultraviolet spectrum (UVC range (100-280 nm), UVB range (280-315 nm) and UVC range 315-400 nm)) which can destroy microorganisms, remove noxious odors, cleansing and purifying the air, generate ozone and to reduce the presence of fungus, viruses, parasites and/or bacteria on the device. The device can emit photonic energy in the visible light spectrum (400-720 nm) or in any of the individual color spectrums, including but not limited to, colors in the red, green and blue spectrum or full spectrum white light, all of which can provide benefits for a user as well as enhance the ambiance of a space. Additionally, the device can be used to emit photonic energy in the infrared spectrum for wellness benefits and/or heating. The device can also optionally emit electromagnetism such as, but not limited to, pulsed electromagnetic field energies that can create geomagnetism fields and/or restore or enhance ambient geomagnetic and/or electromagnetic gauss levels.

The device can optionally include selected natural stones such as jade, tourmaline, bian stones, quartz, germanium or crystals; salt blocks, minerals, man-made materials, other natural materials such as, but not limited to, iron, nickel and cobalt, all of which emits photonic energy, natural geomagnetism, combinations thereof and/or that provide wellness benefits. The photonic energy and/or electromagnetic energy emitted from the natural and man-made materials can occur naturally and can be enhanced by heating or applying electric current the material. The electromagnetic energy is emitted the range of 1-100 Hz, where humans emit electromagnetic frequencies in the range of 9-16 Hz; average for males emit electromagnetic energy at about 12.2 Hz, average females emit electromagnetic energy at about 12.8 Hz for females and the mean human average is 12.3 Hz. The combination of electromagnetic energy and heat have been found to be useful in treating musculoskeletal issues, circulation issues, nervous system issues, digestion issues, respiration issues, wounds and other areas of concern. Typical ranges of Hz emitted by the device ranges from about 1 Hz to about 100 Hz and more typically, about 1 Hz to about 30 Hz and even more particularly, about 1 Hz to about 22 Hz.

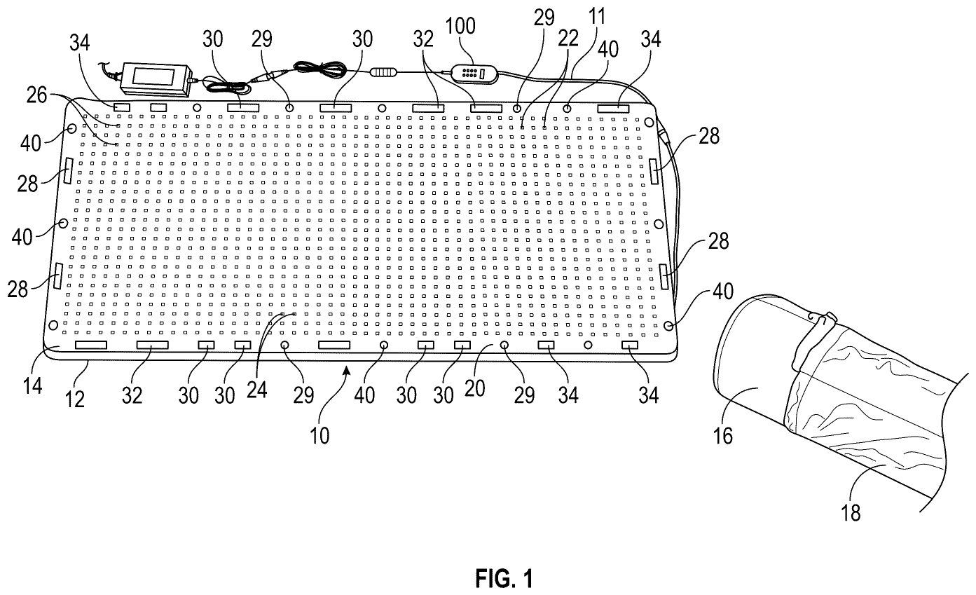

Referring to FIG. 1, a multipurpose device with controllable photonic modalities is illustrated at 10. The multipurpose device 10 includes a substrate 12 having a top or front surface 14 onto which one or more arrays of controllable photonic emitters 20 is attached. The substrate 12 with the one or more arrays of controllable photonic emitters 20 is configured to be rolled into a coil 16 and retained in a sleeve 18 for compact storage and to be unrolled such that the substrate 12 and the array of controllable photonic emitters 20 do not overlap.

The array of controllable photonic emitters 20 includes emitters 22 that emit light in the visible spectrum and optionally the infrared spectrum. The emitters 22 are controllable to emit a selected wavelength corresponding to a color. While all colors in the visible spectrum can be emitted, the emitters 22 can be controlled to emit at least white light, red light, blue light and green light. The array of controllable emitters 20 can also include emitters 24 that emit photonic energy in the infrared spectrum and optionally emitters 26 that emit photonic energy in the ultraviolet spectrum

In some embodiments, the device 10 includes electromagnetic energy emitters, including but not limited to, one or more pulsed electromagnetic field motor 28, that are configured to emit pulsed electromagnetic frequency energy. The pulsed electromagnetic frequency energy creates magnetic fields that restore or enhance magnetic flux levels that can be beneficial to a user's wellness. In some embodiments, the device 10 includes natural materials 29 that emit electromagnetic energy such as, but not limited to, iron, nickel and cobalt.

The device 10 can also optionally include natural stones 30 that emit photonic energy. In other embodiments, the device 10 can include salt mineral blocks 32 to trace minerals and provide halotherapy that ionizes and cleans the ambient air. In yet other embodiments, the device 10 can include man-made materials 34 that emit photonic and/or electromagnetic energy.

The inclusion of the infrared emitters 24, the ultraviolet emitters 26, the pulsed electromagnetic field motors 28, the natural stones 30, the salt blocks 32 and the man-made stones 34 are optional by themselves or in combination to form different applications. For instance, when the device 10 is used for a mat, the device can include visible light emitters 22 and infrared emitters 24. When the device is hung from a wall or door, the infrared emitters 24, the ultraviolet emitters 26, the pulsed electromagnetic field motors 28, the natural materials 29 that emit biomagnetic energy, the natural stones 30, the salt mineral blocks 32 and the man-made stones 34 can be included as the user will not be in contact with the device 10.

The device 10 includes a plurality of grommets 40 located about the perimeter 42. The grommets 40 can be used to hang the device from a surface. Alternatively, the grommets 40 can used to retain a substantially optically transparent and substantially water impermeable plastic cover on the device to prevent sweat from permeating the substrate.

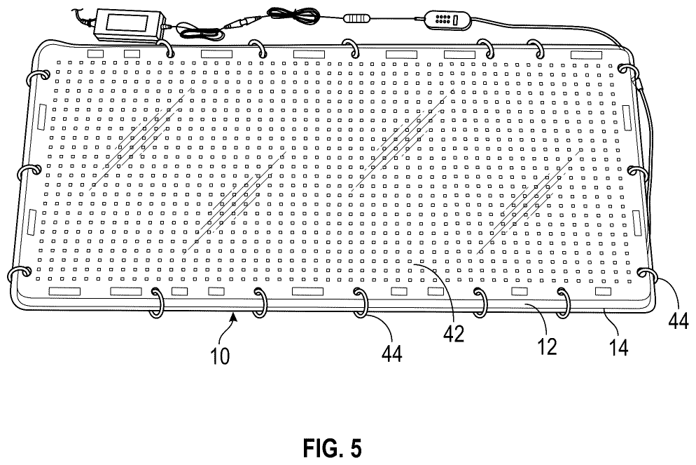

Referring to FIGS. 1 and 5, the device 10 is configured for use as a mat on a floor surface. The substrate 12 for use as a mat includes closed cell foam or tightly woven cloth material. The substrate 12 of closed cell foam or tightly woven cloth material provides cushion and comfort to the user, whether the user is passively sitting or laying on the mat or actively exercising, performing yoga, meditating or receiving a massage. Additionally, the substrate 12 is durable and resistant to wear, which can extend the useful life of the device. Referring to FIG. 5, a plastic film 42 that is substantially optically transparent and substantially water impermeable plastic cover can be placed onto the top surface 14 and retained with ties 44 to retain the plastic film 42 on the substrate 12. An exemplary material of construction for the plastic film 42 is polyvinylchloride (PVC), however other plastic materials are also within the scope of the present disclosure. The plastic film 42 is used to prevent perspiration or other liquids from permeating the substrate 12 to maintain hygiene in the device 10. In some instances, the film 42 is configured to be disposable with an intended one-time use.

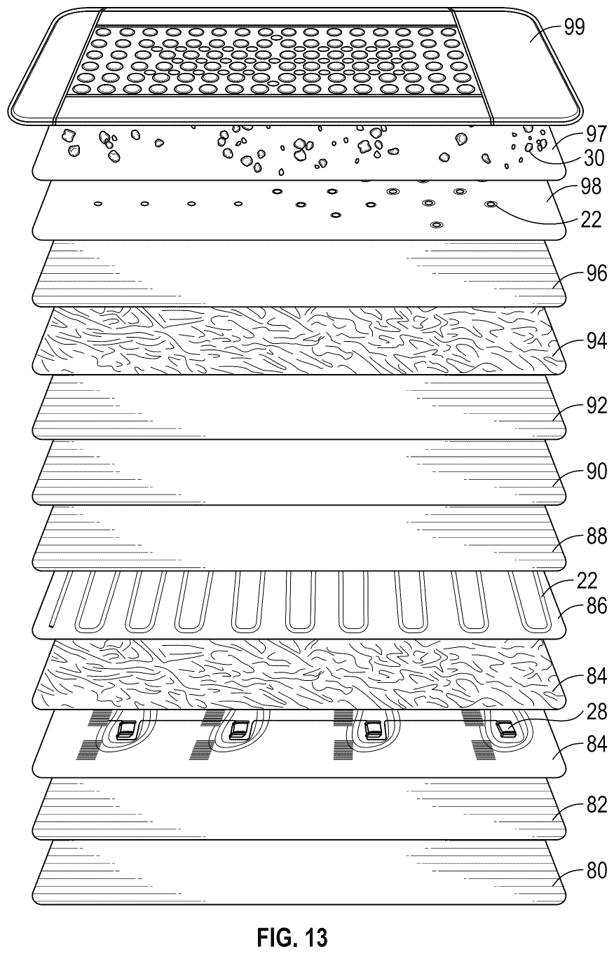

Referring to FIGS. 1 and 13, the device 10 includes a plurality of layers that are designed to cause electromagnetic energy, IR energy and visible light energy to be emitted through a top layer 99. The device 10 includes a bottom layer 80 that is substantially moisture and wear resistant, so that the bottom layer prevents moisture from absorbing into the interior of the device and damaging the electronic equipment. The bottom layer 80 is wear resistant to prevent the bottom layer 80 from eroding due to wear with a surface as the user is engaging the top layer 9.

The device includes an intermediate layer 82 that is positioned between the bottom layer 80 and a PEMF system layer 84 to which the electromagnetic field motors 28 are secured. The insulating layer 82 is substantially non-conductive to prevent electric energy from electromagnetic field motors 28 to conduct to ground. Rather, the PEMF energy from the electromagnetic field motors 28 is directed upwardly. An exemplary and non-limiting material of construction for the insulating layer 82 is a cotton sheet. However, other non-conductive are also within the scope of the present disclosure.

The device 10 includes a thermal insulating layer 84 that is positioned above the PEMF system layer 84. The thermal insulating layer 84 is located below a thermal layer 86 that retains the IR emitters 22. The thermal insulating layer 84 substantially prevents thermal energy from being directed toward the PEMF system layer 84 and protects the electromagnetic field motors 28 from the heat generated by the IR emitters 22. As illustrated the IR emitters 22 includes a resistance heating wire. However, other IR emitters are within the scope of the present disclosure. An exemplary, non-limiting material of construction for the thermal insulating layer 84 includes a thermal foil insulation.

The layer 88 above the thermal layer 86 includes a sensor that senses the temperature created by the IR emitters 22 so that a controller can adjust the amount of current through the IR emitter 22 to control the temperature experienced by the user.

A layer 90 is configured to allow heat to raise from the thermal layer 86, but has some insulating effects such that the user does not experience excessive heat.

A fireproof layer 92 is positioned above the layer 90 that prevents against user experiencing excessive temperatures. The fireproof layer 92 prevents the user from experiencing flames in the event a fire is caused below by the IR emitters 22 or a malfunction of the electromagnetic field motors 28. An exemplary, non-limiting, material of construction of the fireproof layer 92 is a fireproof cotton layer.

An upper insulating layer 94 is above the fireproof layer 92. The upper insulating layer 92 allows heat to gradually pass therethrough such that the temperature experienced by user engaging the top layer 99 gradually raises as the user is on the device 10. An exemplary, non-limiting material of construction of the upper insulating layer 92 is a thermal foil insulation.

A waterproof layer 96 is above the upper insulating layer 94. The waterproof layer 98 prevents moisture, such as sweat or spilled liquid, from affecting the insulating layers and/or the electronic equipment, including the IR emitters 22 and the electromagnetic field motors 28. An exemplary, non-limiting material of construction of the waterproof layer 96 is polyvinylchloride polymer.

A layer 98 is above the waterproof layer 96. The layer 98 is typically non-conductive and retains visible light emitters 22. The visible light emitters 22 are typically light emitting diodes (LED) that are controllable between a plurality of colors of the spectrum and also white light, such that the user can experience the desired wavelengths. In other embodiments, the plurality of LEDS is configured to emit a selected wavelength.

The layer 97 is above the layer 98 and retains the natural stones 30 and optionally man-made stones 34. The natural stones 30 are located above the IR emitters 22 such that the IR emitters 22 heat the natural stones 30 which causes the natural stones 30 to emit photons that are beneficial to the user.

The upper layer 99 is above the layer 97 and is constructed of a material that is comfortable to the touch of the skin to provide comfort to the user. The upper layer 99 also includes a transparent area that allows visible light to pass therethrough to provide phototherapy to the user. An exemplary material of construction of the upper layer 99 besides the transparent area is a polyurethane based material.

When used as a mat, the array of controllable photonic emitters 20 includes emitters 22 that emit light in the visible spectrum and optionally the infrared spectrum. Other prior mentioned photonic modalities including the ultraviolet emitters 26, the pulsed electromagnetic field motors 28, the natural materials 29 that emit electromagnetic energy, the natural stones 30, the salt mineral blocks 32 and the man-made materials 34 can optionally be included in the 10 and supported by the substrate 12 to provide additional photonic and electromagnetic benefits and effects. When used as a mat, the device 10 typically has a length of at least about 5 feet and a width of at least about 2.5 feet. However, other length and width dimensions of the device are within the scope of the present disclosure.

Referring to FIGS. 1 and 13, an exemplary, non-limiting construction of the device 10 includes a plurality of layers that are designed to cause electromagnetic energy, IR energy and visible light energy to be emitted through a top layer 99. The device 10 includes a bottom layer 80 that is substantially moisture and wear resistant, so that the bottom layer prevents moisture from absorbing into the interior of the device and damaging the electronic equipment. The bottom layer 80 is wear resistant to prevent the bottom layer 80 from eroding due to wear with a surface as the user is engaging the top layer 9.

The device includes an intermediate layer 82 that is positioned between the bottom layer 80 and a PEMF system layer 84 to which the electromagnetic field motors 28 are secured. The insulating layer 82 is substantially non-conductive to prevent electric energy from electromagnetic field motors 28 to conduct to ground. Rather, the PEMF energy from the electromagnetic field motors 28 is directed upwardly. An exemplary and non-limiting material of construction for the insulating layer 82 is a cotton sheet. However, other non-conductive are also within the scope of the present disclosure.

The device 10 includes a thermal insulating layer 84 that is positioned above the PEMF system layer 84. The thermal insulating layer 84 is located below a thermal layer 86 that retains the IR emitters 22. The thermal insulating layer 84 substantially prevents thermal energy from being directed toward the PEMF system layer 84 and protects the electromagnetic field motors 28 from the heat generated by the IR emitters 22. As illustrated the IR emitters 22 includes a resistance heating wire. However, other IR emitters are within the scope of the present disclosure. An exemplary, non-limiting material of construction for the thermal insulating layer 84 includes a thermal foil insulation.

The layer 88 above the thermal layer 86 includes a sensor that senses the temperature created by the IR emitters 22 so that a controller can adjust the amount of current through the IR emitter 22 to control the temperature experienced by the user.

A layer 90 is configured to allow heat to raise from the thermal layer 86, but has some insulating effects such that the user does not experience excessive heat.

A fireproof layer 92 is positioned above the layer 90 that prevents against user experiencing excessive temperatures. The fireproof layer 92 prevents the user from experiencing flames in the event a fire is caused below by the IR emitters 22 or a malfunction of the electromagnetic field motors 28. An exemplary, non-limiting, material of construction of the fireproof layer 92 is a fireproof cotton layer.

An upper insulating layer 94 is above the fireproof layer 92. The upper insulating layer 92 allows heat to gradually pass therethrough such that the temperature experienced by user engaging the top layer 99 gradually raises as the user is on the device 10. An exemplary, non-limiting material of construction of the upper insulating layer 92 is a thermal foil insulation.

A waterproof layer 96 is above the upper insulating layer 94. The waterproof layer 98 prevents moisture, such as sweat or spilled liquid, from affecting the insulating layers and/or the electronic equipment, including the IR emitters 22 and the electromagnetic field motors 28. An exemplary, non-limiting material of construction of the waterproof layer 96 is polyvinylchloride polymer.

A layer 98 is above the waterproof layer 96. The layer 98 is typically non-conductive and retains visible light emitters 22. The visible light emitters 22 are typically light emitting diodes (LED) that are controllable between a plurality of colors of the spectrum and also white light, such that the user can experience the desired wavelengths. In other embodiments, the plurality of LEDS is configured to emit a selected wavelength.

The layer 97 is above the layer 98 and retains the natural stones 30 and optionally man-made stones 34. The natural stones 30 are located above the IR emitters 22 such that the IR emitters 22 heat the natural stones 30 which causes the natural stones 30 to emit photons that are beneficial to the user.

The upper layer 99 is above the layer 97 and is constructed of a material that is comfortable to the touch of the skin to provide comfort to the user. The upper layer 99 also includes a transparent area that allows visible light to pass therethrough to provide phototherapy to the user. An exemplary material of construction of the upper layer 99 besides the transparent area is a polyurethane based material.

Once the stack of layers is constructed, the edges of at least the lower layer 80 and the upper layer 99 are secured together to retain the stack of layers in alignment. In some embodiments, the edges are sewn together and in other embodiments that edges are bonded together.

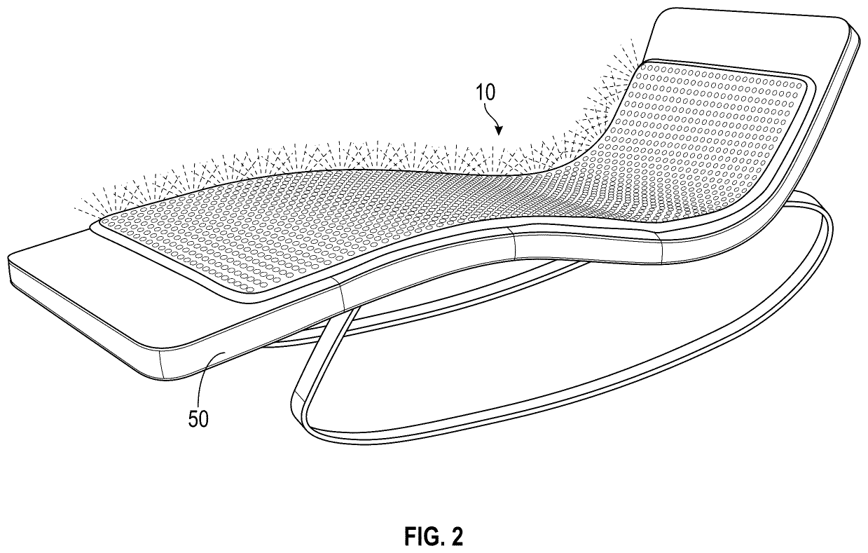

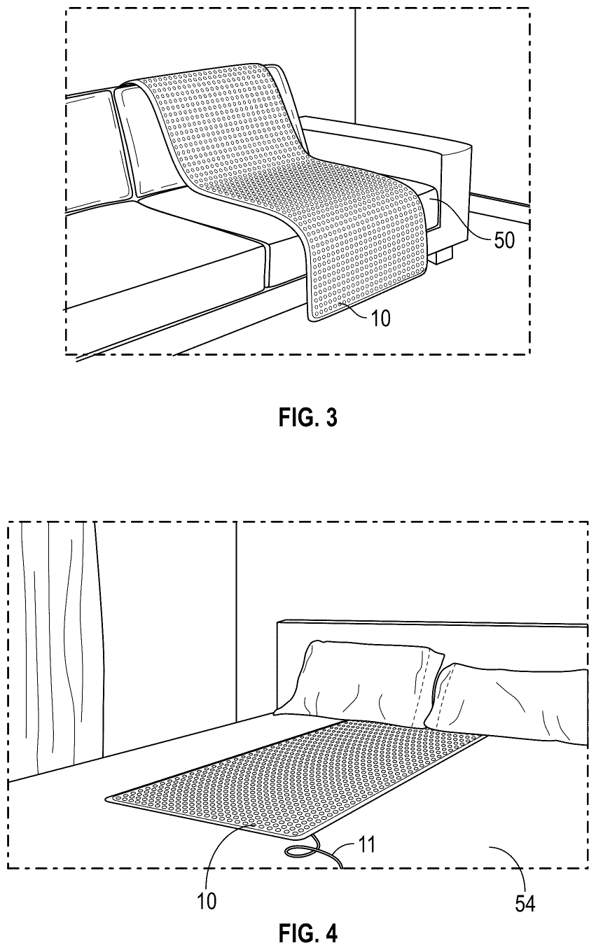

Referring to FIGS. 2-4, the device 10 is used as cover or sheet for the person to sit or lay upon, where the area of the device 10 is determined based upon the required area needed to provide the treatment. Referring to FIG. 2, the device 10 is used as a cover for a lounging chair 50. When the user reclines on the lounging chair 50 the device 10 emits controlled photonic energy from the array of photonic emitters 20, the natural stones 30 and/or the man-made stones 34. The device can additionally and/or alternatively provide electromagnetic energy from the pulsed electromagnetic field motors 28 and/or the natural materials 29 that emit electromagnetic energy. Similarly, referring to FIG. 3, the device 10 can be draped over a couch 52 or other sitting furniture or appliance where the user sits on the device 10 to receive controlled photonic energy and/or electromagnetic energy. Referring to FIG. 4, the device 10 can also be used as a sheet or spread on a bed 54.

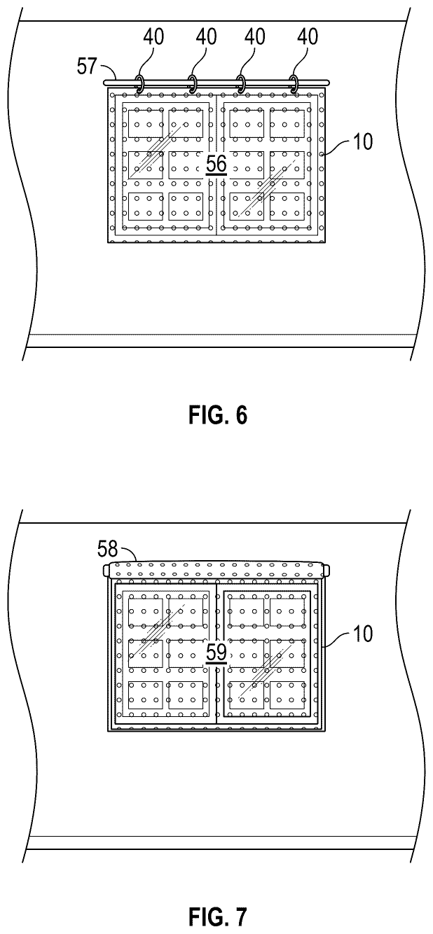

The device 10 can also be hung in numerous locations within a location to provide a person with the controlled photonic energy and/or electromagnetic energy, or to change the environmental conditions and/or to provide ambiance. Referring to FIG. 6, the device 10 can be hung over a window 56 with a rod 57 to provide the controlled photonic energy and/or electromagnetic energy while functioning as a curtain. Referring to FIG. 7, the device 10 can be coiled about a spring biased roller 58 to function as a shade or blind over a window 59, where the device can be extended to allow the person with the controlled photonic energy and/or electromagnetic energy, or to change the environmental conditions and/or to provide ambiance and to be retracted to allow natural light and/or geomagnetism into the space.

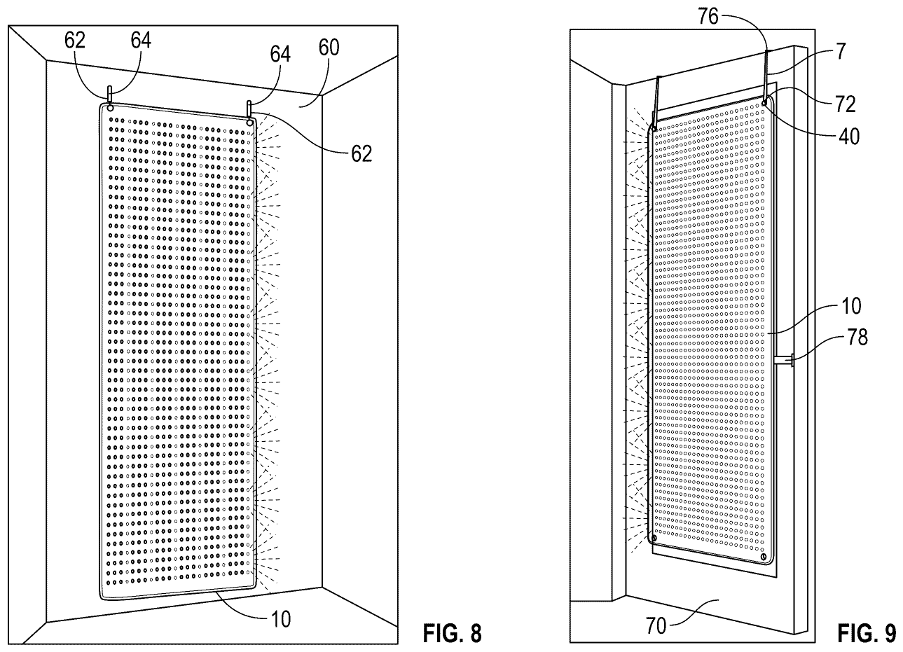

Referring to FIG. 8, the device 10 can be hung from a wall 60 using loops or carabiners 62 positioned through the grommets 40 where the carabiners or loops 62 attached to hooks 64 extending from the wall 60. Referring to FIG. 9, the device 10 is illustrated being attached to a door 70. Bottom hook portions 72 of a retaining member 74 are positioned through the grommets 40 and top hook portions 76 extend over the top of the door 70 to retain the device 10 in an elevated position on the door 70. Optional straps 78 secured to the side edges of the device 10 can be used to retain the device 10 to the door 70 as the door 70 is opened and shut.

The device 10 can be used to provide controlled photonic energy and/or electromagnetic energy to a user by having the user stand proximate the device 10, as illustrated in FIGS. 6-9. Alternatively, the device can be used to control and or modify the ambient conditions in a space, including but not limited to emitting visible light to change the lighting, emitting infrared energy to provide heat, emitting ultraviolet energy to destroy microorganisms, remove noxious odors, cleanse and purify the air and/or generate ozone. As the device 10 is used as a hanging device with little to no contact with the user, the substrate 12 can be less robust than a device used by the user for sitting or reclining. The hanging device 10 can utilize a less heavy tightly woven fabric or a sheet of cloth or plastic material to support the elements emitting photonic energy, electromagnetic energy and/or halotherapy.

The size of the hanging device 10 can be varied depending upon its use. When hung from the door 70, the area of the device 10 should be less that the area of the door 70. However, when hung from a wall 60, the size of the device can vary from the size used as a mat to provide photonic energy, electromagnetic energy and/or halotherapy to a user to the area of the wall when used to modify ambient conditions in the space.

Additionally, the device can include one or more of the mentioned photonic modalities and/or the mentioned electromagnetic modalities by themselves or in combination to provide the desired treatment for the user or modify the ambient conditions in the space. The photonic modalities for use in the hanging device 10 include the controllable visible light emitters 22, the infrared emitters 24, the ultraviolet emitters 26, the natural stones 30, the salt blocks 32 and the man-made stones 34 by themselves or in any desired combination while the biomagnetic modalities include the pulsed electromagnetic field motors 28 and/or the natural materials 29.

The device 10 is powered by a standard 110 volt power supply and includes a cord 11 that has a plug that plugs into a standard electrical outlet. The cord 11 is sufficiently long to provide a desired range of movement to place or hang the device 10 in a selected location.

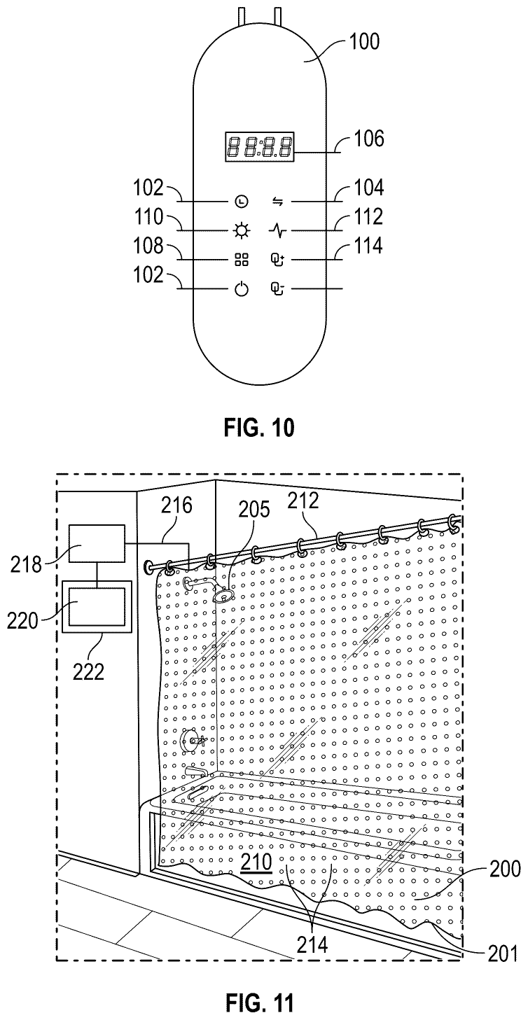

The device 10 includes a corded or battery-operated controller 100 as illustrated in FIGS. 1 and 10. The controller 100 includes an on/off button 102 that allows the user to operate the device 10. The controller 100 includes a timer function 102 that allows the duration of use to be set. A shift button 104 allows the user to select one of a plurality of preset times, including for instance, 10 minute intervals starting at 10 minutes and ending at 60 minutes. A screen 106 allows the user to visual the control adjustments being made. The button 108 allows for the selection of wavelengths in the visible spectrum, including but not limited to, red, green, blue and white. The selection is made using the shift button 104 and viewing the screen 106. Typically, at least near infrared photonic energy is emitted with the selected visible photonic energy. Once the wavelength is selected, the intensity or brightness button 110 is engaged and the shift button 104 is used to provide the desired intensity. The visible light can be pulsed by selecting the button 112 and utilizing the shift button 104 and screen 106 to control the pulse rate. By way of non-limiting example, the pulse rate can be controlled between 1 and 999 Hz.

The button 114 can be utilized to energize the pulsed electromagnetic field motors 28, where the shift button 104 and screen can be utilized to set the rate and intensity of the electromagnetic pulses. The button 116 can be utilized to energize the ultraviolet emitters 26 where the shift button 104 and the screen 106 are utilized to control the ultraviolet emitters. Other control features can also be added to the controller as needed to provide the selected photonic and/or electromagnetic energy for a selected purpose.

Additionally, the controller 100 can include preset protocols for specific purposes. Table 1 below provides a sample of present protocols that can be utilized.

TABLE 1

| Desired Effect | Time (Min) | Red | Green | Blue | White | NIR | EM (Hz) |
|---|---|---|---|---|---|---|---|
| Cellulite Smoothing | 15 | Y | N | N | Y | Y | Y |
| Skin Toning | 10 | Y | Y | N | Y | Y | Y |
| Body Cleansing | 10 | Y | Y | Y | N | Y | Y |
| Weight Management | 15 | Y | N | Y | N | Y | Y (8-12) |
| Strength and Recovery | 15 | Y | N | N | Y | Y | Y (12) |
| Muscle Relaxation | 30 | Y | N | N | N | Y | Y (10) |
| Energy and Endurance | 30 | N | N | Y | N | Y | Y (12) |
| Relaxation and | 30 | Y | N | N | N | Y | Y (20) |
| Stress Relief | | | | | | | |
| Gentle Cleanse | 30 | N | Y | N | N | Y | Y (7) |
| Flexibility | 15 | Y | N | N | Y | Y | Y (16-20) |
| Toxic Deep Cleanse | 15 | Y | Y | N | N | Y | Y (10) |
| Calm & Clarity | 15 | N | Y | N | Y | Y | Y (14-20) |
| Mood Boost | 15 | N | N | Y | Y | Y | Y |
| Mind Wellness (PM) | 30 | N | N | N | Y | Y | Y |
| Mind Wellness (AM) | 30 | N | N | Y | Y | Y | Y |
| Deep Sleep (PM) | 30 | Y | N | N | N | Y | Y (5) |
| Deep Sleep (AM) | 30 | Y | N | N | Y | Y | Y (5) |

The preset protocols allow the user to efficiently receive the desired controlled photonic energy without having to adjust the protocol for curtained desired effects. It is understood the protocols can include electromagnetic sessions in desired Hertz and time periods along with the photonic treatments or by itself.

Referring to FIG. 11, another embodiment of the device is illustrated at 200. The device 200 is used as a shower curtain that is supported by a shower rod 202 that spans the length of a bath tub 204 or shower area. Loops 206 are positioned through grommets and about the shower rod 202 to suspend the shower curtain 200. The shower curtain is sized to span a length of the bath tub 204 or shower area and has a height where a bottom edge 201 extends below the top of the bath tub 204 or shower area.

The device 200 includes a back substantially water impermeable sheet 210 that can be optically reflective and a front substantially water impermeable sheet 212 that is substantially optically transparent. Arrays of LED light emitters and optional near infrared (NIR) emitters 214 are positioned between the two sheets 210 and 212 and the LED and/or NIR lights 214 are sealed between the two sheets 210 and 212. A cord 216 extends from a top corner of the shower curtain away from a shower head 205 to aid in prevent moisture from engaging the cord 216. The cord 216 is in electrical communication with a control panel 218 that is powered by a battery pack 220 that can have a unique interface with a receptacle 222.

The power in the battery pack is calculated by determining the power needed to power the control panel 218 and the array LED light emitters and/or NIR emitters 214 for a selected period of time. The power in the battery pack is sufficiently low so that no harm would come to a user in the event of a failure of the seal between the sheets 210 and 212.

The control panel 218 allows the user to select the wavelength, intensity and pulsation frequency of the visible light and/or NIR as well as the duration of use. In some embodiments, the device 200 includes the pulsed electromagnetic field motors 205 and/or the natural materials 207 that emit biomagnetic energy. The user can then bathe or shower while received a controlled phototherapy session and/or a biomagnetic session.

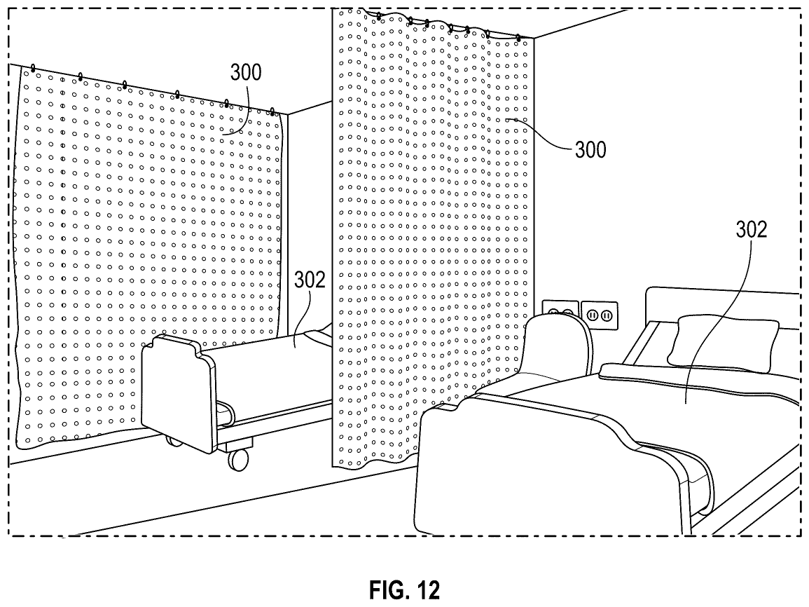

Referring to FIG. 12, another embodiment of the device is illustrated at 300 where the device 300 is used as room divider. The device 300 is positionable about a bed 302 where the device is able to change ambient conditions for a person using the bed 302. The device 300 can be utilized to provide photic energy and/or electromagnetic energy for treatment. The device 300 can also include larger densities of UV emitters such that the devices can be used to sanitize and disinfect the atmosphere in a room, such as a multi-person hospital room, where the UV light may be advantageous is prevent a person from become ill with airborne bacteria, fungi and/or viruses.

Although the subject matter has been described in language specific to structural features and/or methodological acts, it is to be understood that the subject matter defined in the appended claims is not necessarily limited to the specific features or acts described above as has been determined by the courts. Rather, the specific features and acts described above are disclosed as example forms of implementing the claims.

1. A device for providing controlled photonic modalities and electromagnetic modalities, the device comprising:
a substrate having a length and a width;
at least one array of controllable photonic emitters attached the substrate;
at least one electromagnetic energy emitter attached to the substrate; and
a controller configured to allow a user to select the type of photonic energy and the electromagnetic energy.

2. The device of claim 1, wherein the substrate comprises a tightly woven cloth material or a closed cell foam material.

3. The device of claim 1, wherein the substrate comprises a sheet of cloth material.

4. The device of claim 1 wherein the substrate comprises first and second substantially water impermeable sheets and wherein the at least one array of controllable photonic emitter are sealed between the first and second sheets.

5. The device of claim 4, wherein the first sheet is substantially optically reflective and the second sheet is substantially optically transparent.

6. The device of claim 1, wherein the at least one array of controllable photonic emitters comprises an array of controllable LED emitters and/or infrared emitters.

7. The device of claim 1 wherein the substrate is further configured to support one or more of emitters that emit photonic energy in infrared spectrum, emitters that emit photonic energy in the ultraviolet spectrum, one or more pulsed electromagnetic field motor that are configured to emit pulsed electromagnetic frequency energy, natural materials that emit electromagnetic energy, natural stones, salt mineral blocks and man-made materials.

8. The device of claim 1, wherein the device is configured to be used as a mat, a sheet or a cover supported on a surface wherein a user physically engages the device.

9. The device of claim 1, wherein the device is configured to be hung from a door, a wall or a window.

10. The device of claim 9, wherein the hung device is configured to be utilized to provide photonic energy and/or electromagnetic energy to a user.

11. The device of claim 9, wherein the hung device is configured to enhance and/or to modify ambient conditions in a space.

12. The device of claim 4, wherein the device is configured for use as a shower curtain, a room divider and/or an ambient modifier of a space.

13. The device of claim 12, wherein the device is powered by a battery pack.

14. The device of claim 13, wherein the control is preprogrammed with a plurality of protocols.

15. The device of claim 1, wherein the controller is configured to adjust a duration and/or a pulse rate of the photonic energy and/or electromagnetic energy.

16. A device for providing controlled photonic modalities and electromagnetic modalities, the device comprising:
a base layer having an upper surface and a lower surface configured to engage a surface;
a first internal layer supporting pulsed electromagnetic field motor, the first internal layer having a lower surface facing the upper surface of the base layer and an upper surface;
a second internal layer supporting one or more infrared emitters, wherein the second internal layer having a lower surface facing the first internal layer and an upper surface;
a first internal insulating layer between the first internal layer and the second internal layer;
a third internal layer supporting a plurality of visible light emitters, the third internal layer having a lower surface facing the upper surface of the second internal layer
a second internal insulating layer between the second internal layer and the third internal layer;
a fourth internal layer supporting a plurality of stone that emit photons when heated, the fourth internal layer having a lower surface facing the upper surface of the third internal layer and an upper surface; and
an upper layer having a substantially optically transparent area, the upper layer having a lower surface facing the upper surface of the fourth internal layer and an upper surface configured to engaged by a user.

17. The device of claim 16 and further comprising:
a substantially non-conductive layer positioned between the base layer and first internal layer.

18. The device of claim 16 and further comprising:
a temperature sensor positioned the second internal layer and the second internal insulating layer.

19. The device of claim 16 and further comprising:
a substantially waterproof layer positioned between the second internal insulating layer and the third internal layer.

20. The device of claim 16 and further comprising:
a substantially fireproof layer positioned between the second internal layer and the second internal insulating layer.
